(19) United States
(12) Patent Application Publication
(10) Pub. No.: US 2015/0065757 A1
(43) Pub. Date: Mar. 5, 2015

(54) COMPOSITE MATERIAL COMPOSED OF A POLYMER CONTAINING FLUORINE, HYDROPHOBIC ZEOLITE PARTICLES AND A METAL MATERIAL

(71) Applicants: Achim Koch, Moosburg (DE); Michael Zavrel, Olching (DE)

(72) Inventors: Achim Koch, Moosburg (DE); Michael Zavrel, Olching (DE)

(73) Assignee: CLARIANT PRODUKTE (DEUTSCHLAND) GMBH, Frankfurt Am Main (DE)

(21) Appl. No.: 14/125,023

(22) PCT Filed: Jun. 1, 2012
(86) PCT No.: PCT/EP2012/060377

§ 371 Date: Nov. 11, 2014

(30) Foreign Application Priority Data
Jun. 10, 2011 (EP) 11004778.4

(51) Int. Cl. B01J 20/28; B01J 20/16; B01J 20/26; C07C 29/76

(52) U.S. Cl. CPC B01J 20/28026; C07C 29/76; B01J 20/16; B01J 20/261
USPC 568/917; 502/62

(57) ABSTRACT

The invention relates to a composite material comprising a) a porous matrix of a polymer containing fluorine and having a percentage of tetrafluoroethylene monomer units of at least 95 mol % based on the total of monomer units, b) hydrophobic zeolite particles which are embedded in the matrix and around which matrix filaments extend, and c) at least one metal material. The invention further relates to the use of the composite material for adsorbing organic molecules from a gaseous or liquid mixture of substances that contains at least one organic component, and to a method for removing organic molecules from a gaseous or liquid mixture of substances.

### FIELD OF THE INVENTION

The invention relates to a composite material composed of a polymer containing fluorine, hydrophobic zeolite particles and a metal material with improved mechanical and thermal properties for the adsorption of organic molecules of an aqueous fluid.

### BACKGROUND TO THE INVENTION

The separation of liquid or gaseous substance mixtures has been practised commercially and industrially for decades for a plurality of applications. Examples are chromatographic analytical or preparative separation (high pressure liquid chromatography, HPLC or gas chromatography GC) by means of solid phase extraction (SPE), the separation of gases or liquids by pervaporation or vapour permeation with so-called MMMs (Mixed Matrix Membranes), the drying of gases and liquids with porous hydrophilic drying agents and selective adsorption/desorption processes on hydrophobic zeolite. Since the drying agents alone, either in powder or granule form, would not be suitable for certain applications under pressure, with high pressure gradients or mechanical loading, they are generally applied to carriers. The use of moulded bodies to maximise the effective surface with the smallest possible structural form is therefore prior art. These are generally membranes which have been made up of composites of sorptively acting materials together with polymers.

Polytetrafluoroethylene (PTFE, produced e.g. by DuPont or Dyneon—3M) is suitable as a matrix polymer for this purpose, especially as it can be fibrillated, is thermally stable, chemically inert and hydrophobic, i.e. it can be processed to form stable and highly flexible fibre fleeces, can be used in the working temperature range of −250 to +260° C., neither absorbs water nor is soluble; in addition, PTFE is largely inert with respect to acids and lyes.

PTFE is partially crystalline and can be fibrillated above the phase transition temperature of 19° C., i.e. by applying shearing forces to PTFE power, granules or the PTFE balls contained in the dispersion, the crystallites contained in the material can be uncoiled to form thin filaments (this effect can be observed even better above 30° C.; this is where the second phase transition of PTFE takes place). These filaments, in the best case only a few molecule layers thick, are capable, using an appropriate processing technique, of extending around, embedding and holding large quantities of filler, by means of which high-grade cross-linked, highly filled PTFE filler composites are obtained. Moreover, the polymer fibres hook and loop onto one another during the shearing, and this gives the composite material a certain degree of mechanical stability. The ability to be processed into films and moulded bodies can, however, be greatly hindered by the strong deformation forces required depending on the filler, and this is why it has proved best to use, wherever possible, lubricants (water, alcohols, crude oil distillates, hydrocarbons and other solvents) which facilitate the processing process, support the fibrillation and prevent premature destruction/tearing of the fibres due to excessive shearing. After the shaping, the solvent that has been added is generally eliminated by heating, by means of which an additional defined degree of porosity remains. By means of an optional sintering process of the PTFE material at temperatures of around 330° C., but below 360° C. (start of decomposition) the composite material obtains its final stability and shape.

The embedding of fillers into fibrillated PTFE is known and is described in various patent documents.

The prior art patent documents can be divided up as follows depending on the type of zeolite described: JP 04048914 describes the production of a film with a moisture extracting function composed of 1-10 parts fibrillatable fluoropolymer (i.e. PTFE) and 100 parts moisture absorbent filler (calcium chloride, zeolite, aluminium oxide or silica) with particles sizes <50 μm, produced by kneading and the production of films. An auxiliary agent (i.e. water, alcohol), which supports the fibrillation process, is optionally used.

EP 1396175 describes a self-supporting moisture-absorbent film made of ultra-high molecular (UHMW) polymer (MW>1,000,000 g/mol) with embedded drying agent for protecting electroluminescent elements from moisture.

JP 63028428 describes a drying agent composed of PTFE, zeolite and liquid lubricant which is converted into moulded bodies by kneading and rolling out. Moreover, a hygroscopic metal or alkali metal salt (e.g. lithium chloride) is separated from its ethanolic solution on the surface of the zeolite particles, and then the material is dried. It is also described how rolled films provided with indentations can allow vapour to pass in the longitudinal direction.

EP 0316159 describes the production of moulded bodies for catalysis or absorption purposes made of PTFE powder or dispersion and sorptively effective filler in the ratio 1:0.5-10, optionally with the use of a liquid lubricant, by mixing the components to form a paste and then sintering at >327° C., by means of which a self-supporting moulded body with a surface area >50 m²/g is produced.

WO 2005/049700 describes a method for producing PTFE filler composites based on active filler (=“shearing material”) and 0.1-20% w/w polymer particles (with dimensions shearing material >1 μm to polymer particles <1 μm 5:1 to 2000:1). The method comprises the steps: (a) dispersing the material while mixing intensively to form a doughy mass; (b) rolling out the mass to form a mat; and (c) folding the mat and rolling out at an angle of 45-135° in relation to the previous direction, excess solvent being removed before or after the mixing step, and all of the steps being carried out at 15-40° C. The composite material can be used as an adsorption film for gases and liquids.

EP 0659469 describes a membrane composed of PTFE and zeolite A for the separation of liquids, e.g. water/ethanol by means of pervaporation or vapour permeation. The membrane is separated on a porous carrier. The hydrophilic zeolite used has a SiO₂:Al₂O₃ ratio of 2-6:1.

JP 2010000435 describes a composite membrane composed of fluoropolymer (=porous carrier, e.g. PTFE) and crystalline zeolite which, after surface treatment (metallation or hydrophilisation) of the carrier is applied to the carrier by a hydrothermal method in order to separate liquids (e.g. water/ethanol) or to extract residual water from >95% ethanol. The secure anchoring of the zeolite on the fluoropolymer surface is achieved according to the invention despite the actual incompatibility of the two substances, and so improves the resistance of the composite membrane. In addition, JP 2010115610 describes the introduction of high molecular polymers (silicone rubber, polyvinyl alcohol, polyacrylamide, polyethylene glycol, polyimide or polyamidimide or its carbides) at defective points of the crystalline zeolite surface in order to improve the separating capacity of the aforementioned composites.

U.S. 20100038316 describes a composite body made of PTFE and zeolite which is produced by a binder and/or zeolite being applied to a PTFE layer and so both components being connected (“bonded”) to one another layer by layer. This produces at least 2, at most 3 layers which together produce a membrane or a film from the components PTFE, adhesive and zeolite. The adhesive (or binder) can e.g. be polyvinyl alcohol which dissolves in an appropriate solvent, is mixed with the zeolite and is then applied to the PTFE layer. The zeolite can also be changed such that it is catalytically effective, e.g. by metal ions.

WO 07104354 describes a packing structure for column chromatography (e.g. HPLC) which is used for the separation of various components from a fluid. The filling consists of an elastic polymer network in which the actual filler material is embedded. The elastic network consists of at least one of the following substances: organic or inorganic material, polymer, rubber, caoutchouc, PTFE, expanded PP etc., and the actual filler material in turn consists e.g. of zeolite and/or PTFE. Moreover, a support element is claimed which the filler material can surround or which can be located above, below or on both sides of the filler material. The supporting element can be a stainless steel wire sieve, fabric, sintering body or filter. The use of a wire sieve is described as advantageous when removing compacted packing material at the top of the HPLC column and when producing the column.

EP 0 773 829 describes composite membranes made of fibrillated PTFE or blown microfibres (polyamide, polyester, polyurethane, polyolefin etc.) to which, with the aid of a liquid lubricant to simplify the processability, a hydrophobic molecular sieve with a pore diameter of 5.5-6.2 angstrom in a ratio of 40:1 to 1:40 is added as a selective sorption medium exclusively for the solid phase extraction or chromatographic separation. The doughy mass is calendered biaxially, by means of which finally, after drying, a porous film is produced the porosity of which can be derived from the amount of lubricant. Solid phase extraction is understood to be the physical separating process between a fluid and a solid phase, the component to be isolated (=analysed) being dissolved in a solvent. A thermal desorption step of the adsorbed organic component produced is furthermore claimed as part of the method. In the aforementioned patent, among others the patents U.S. Pat. No. 4,153,661, U.S. Pat. No. 4,460,642 and U.S. Pat. No. 5,071,610 of the same Patentee are cited which similarly describe porous fibrous membranes based on PTFE for the inclusion of sorbents or catalytically active particle-like substances.

The present invention is intended to make available a material which is suitable for adsorbing organic molecules from fluids (i.e. gases and liquids) and thereby has improved material stability and increased life in technical column packings, as well as a high adsorption capacity for the organic target molecules, i.e. a high adsorption capacity, i.e. the capability of adsorbing a high mass of organic target molecules per mass unit of adsorption material with at the same time low water adsorption. Of particular significance for the technical application are the lowest possible reduction in pressure by the arrangement of the material according to the invention in a fluid flow and improved thermal properties with respect to the materials described in the prior art.

In addition, the invention is intended to make available a method for removing organic molecules from a gaseous or liquid mixture of substances.

This object is achieved by the invention described below.

### DESCRIPTION OF THE INVENTION

In a first aspect the invention provides a composite material comprising
a) a porous matrix composed of a polymer containing fluorine having a percentage of tetrafluoroethylene monomer units of at least 95 mol % based on the total of monomer units;
b) hydrophobic zeolite particles which are embedded in the matrix and around which the latter extends, and
c) at least one metal material;
d) optionally at least one further component,
the amount of metal material c) being 1 to 90% by weight based on the total weight of all of the components,
the ratio of the weight of component a) to the total weight of components b) and d) being 2:98 to 30:70, and
the ratio of the weight of component b) to the weight of component d) being 80:20 to 100:0.

In a second aspect the invention provides a method comprising the steps
a) bringing a mixture of substances containing at least one organic component into contact with the composite material according to the first aspect described above so that the at least one organic component is adsorbed on the composite material and a charged composite material is obtained;
b) separating the mixture of substances and the charged composite material;
c) desorbing the at least one organic component from the charged composite material.

Preferred embodiments are described below and defined in the dependent claims

### Composite Material According to the Invention

In the following components a), b), c) and d) of the composite material according to the invention are described.

The composite material according to the invention comprises a fibrillatable polymer containing fluorine, preferably PTFE, and a hydrophobic zeolite which is suitable for adsorbing small organic molecules of an aqueous fluid. In order to stiffen the material, metal in the form of metal lattice, fabric or netting, perforated or pierced metal plates is added. Surprisingly, it has been found that by adding metal, in addition to mechanical stabilisation, a not inconsiderable positive change to the thermal properties of the material is achieved.

### a) Matrix Composed of Polymer Containing Fluorine

The matrix of the composite material is composed of polymer containing fluorine, i.e. a homo- or copolymer having a percentage of tetrafluoroethylene monomer units of at least 95 mol %. The polymer containing fluorine can be fibrillated and can form a porous matrix by fibrillating. Moreover, the polymer containing fluorine is chemically inert and is not capable of swelling in the presence of water or organic molecules. Preferably, the polymer containing fluorine has a percentage of tetrafluoroethylene monomer units of at least 99 mol %.

Polytetrafluoroethylene (PTFE), tetrafluoroethylene hexafluoropropylene copolymer, tetrafluoroethylene chlorotrifluoroethylene copolymer, tetrafluoroethylene perfluoro-(2,2-dimethyl-1,3-dioxol)-copolymer and tetrafluoroethylene perfluoro (C₁-₆-alkylvinyl ether)-copolymer such as for example tetrafluoroethylene-perfluoro(butenylvinylether)-copolymer can be specified as examples of polymer containing fluorine. PTFE is preferred.

The polymer can be used as a powder or as a dispersion. Surfactant-free PTFE powders are preferably used because the absence of any surface-active substances required for the stability of PTFE dispersions eliminates the undesired effects of the reduction of the available zeolite surface and increase of the water adsorption by such surfactants.

According to the methods described in EP 0 773 829 B1 (and the prior art documents cited in the latter) these polymers can be fibrillated, and so a porous and fibrous matrix is formed.
b) Hydrophobic Zeolite Particles

For the material according to the invention, the sorbents which are suitable for sorbing organic polar molecules from fluids containing water and desorbing them again under appropriate conditions in order to enrich or purify them are of particular interest.

Particularly suitable are hydrophobic zeolites, i.e. zeolites with a molar SiO₂:Al₂O₃ ratio greater than 100:1, preferably greater than 200:1, more preferably greater than 500:1. These zeolites are generally very suitable for the adsorption of organic molecules such as alcohols (e.g. ethanol, butanol), ethers, ketones (e.g. acetone), aldehydes (e.g. acetal dehyde), carboxylic acids (e.g. acetic acid) and carboxylic acid esters (e.g. ethyl acetate) etc. The SiO₂:Al₂O₃ ratio is determined by X-ray fluorescence spectroscopy (XRF) of a sample dried for one hour at 100° C., which is then pressed with a binding agent to form a tablet, by determining the molar ratio of Si:Al which is converted to the molar ratio SiO₂:Al₂O₃.

In order to have particularly good adsorption properties, i.e. to be able to adsorb a large number of organic molecules per unit weight of zeolite, the zeolites should have a large surface area per unit weight determined by the BET method. Zeolites suitable for the present invention have a surface area according to the BET method of 150 m²/g or larger, preferably 200 m²/g or larger, and more preferably of 300 m²/g or larger.

The surface area is determined by a fully automatic ASAP 2010 type nitrogen porosimeter made by the company Micromeritics using nitrogen as the adsorbed gas according to the following method according to DIN 66131 (July 1993). The sample is cooled in a high vacuum to the temperature of liquid nitrogen. Next nitrogen is continuously metered into the sample chambers. By recording the amount of adsorbed gas as a function of pressure, an adsorption isotherm is determined at constant temperature. In a pressure equalisation the analysis gas is removed step by step and a desorption isotherm is recorded. The data according to DIN 66131 (July 1993) are analysed to determine the specific surface area and the porosity according to the BET theory.

From these points of view zeolites of the silicalite, B zeolite, mordenite, Y zeolite, MFI zeolite, ferrierite (FER zeolite), dealuminated, ultrastable zeolite Y (USY zeolite) and erionite (ERI zeolite) types are preferred. The method according to the invention also allows mixtures of these zeolites.

Zeolite particles with a particle size (d₅₀) of 0.5 to 100 μm, more preferably of 1 to 50 μm and particularly preferably of 5 to 25 μm are preferably used. Basically, as the particle size decreases the specific surface area, i.e. the surface area per unit mass increases. A large specific surface area generally leads to a high and so advantageous adsorption speed. Since, however, the handling and processing of a powder becomes increasingly difficult and complex as the particle size decreases, it is not advantageous to choose small particle sizes although this is possible in principle.

A single zeolite type or a mixture of a number of zeolite types can be used. The single zeolite type or the zeolite types can be used in a uniform particle size or in a number of particle sizes.

### c) Metal Material

Suitable for the composite material according to the invention are metal materials, i.e. pure metals and alloys, which are chemically inert in the presence of water and organic molecules, i.e. do not react, or only react to a limited degree with water and/or organic compounds. Limited reaction with water and/or organic compounds means, for example, that passivation of the surface of the metal material occurs, but not a chemical reaction which ultimately leads to total degradation of the metal material.

From these points of view, corrosion-free metals, particularly preferably stainless steels which are used in the food and chemical industry, e.g. X2CrNi1911 (material number 1.4306), X12CrNi177 (material number 1.4310), or X5CrNi1810 (material number 1.4301) are preferred.

The form in which the metal material is present in the composite material is not limited. For example, the metal material can be present in two-dimensional form, i.e. for example in the form of metal lattices, fabrics, nettings or of perforated or pierced metal plates or sheets, or in particle form, i.e. for example in the form of powders or shavings. By means of the structures specified as examples it is guaranteed that a good connection between the metal and the composite material is achieved. The metal material can be present in the composite material in a number of forms, i.e. both in particle form and in two-dimensional form.

When using the metal material in two-dimensional form a mesh width or hole opening of 0.5-5 mm, in particular 1-2 mm is preferred. The number and distribution of holes per surface unit is not especially restricted and is determined by considerations of the person skilled in the art with regard to the desired permeability and stability. Likewise, the thickness of the metal material in the two-dimensional form used is not especially restricted provided that the desired dimensional stability is achieved. For this purpose the thickness of the metal material is customarily 0.1-1 mm, preferably 0.2-0.5 mm, and particularly preferably 0.25 mm

In the composite material according to the invention the amount of metal material c) is 1 to 90% by weight based on the total of all of the components of the composite material. Preferably, the amount of metal material c) is 5 to 80% by weight, more preferably 10 to 70% by weight, and most preferably 15 to 65% by weight based on the total of all of the components of the composite material.
d) Further components

In the composite material according to the invention one or a number of components can optionally be provided which can be chosen, for example, from auxiliary substances, surfactants, lubricants, precipitated silicic acid, silica, activated carbon, pigments, glass beads or fibres, synthetic fibres, fibres of natural origin, clay minerals such as for example bentonite.

The polymer containing fluorine a) is in a ratio to the overall weight of the hydrophobic zeolite particles b) and the optionally provided further component d) of 2:98 to 30:70, preferably of 4:96 to 20:80, and more preferably of 5:95 to 15:85.

The ratio of the weight of the hydrophobic zeolite particles b) to the weight of component b) is 80:20 to 100:0, i.e. component d) is optional. Preferably, the ratio of the weight of the hydrophobic zeolite particle b) to the weight of component d) is 90:10 to 100:0, and more preferably 95:5 to 100:0.

In a preferred embodiment the ratio of the weight of the polymer containing fluorine a) to the overall weight of the hydrophobic zeolite particle b) and the optionally provided further component d) is in a range of 4:96 to 20:80, more preferably 5:95 to 15:85, the ratio of the weight of the hydrophobic zeolite particle b) to the weight of component d) being 90:10 to 100:0.

### Production of the Composite Material According to the Invention

The composite material is produced by mixing components a), b), the optional further component(s) d) and the metal material c) if the metal material c) is used in an appropriate small-part form, i.e. for example in powder form, in the amounts specified above and then by kneading, the fibrillation of the polymer and addition of the zeolite to the porous polymer matrix ensuing upon shearing [FIG. 1]. The kneading is carried out at room temperature or preferably at an increased temperature such as for example 30° C. or more, 50° C. or more or 70° C. or more because at a temperature in these ranges better processability and in particular better fibrillation of the polymer containing fluorine is generally possible. The upper temperature limit is first and foremost determined by thermal stability of the components contained in the mixture. From this point of view processing at a temperature of no more than 200° C., and more preferably of no more than 150° C. is generally preferred.

In order to achieve good miscibility of the components of the composite material, polymer a) and the zeolite b) are preferably used in powder form. The polymer a) can for example also be used in the form of a commercially available dispersion in water. These commercially available dispersions can contain auxiliary substances such as for example stabilisers, surfactants or other components that change the surface tension and/or other auxiliary substances.

In order to facilitate the mixing and shearing process, water or alcohol can be added as lubricants. In order to be able to largely dispense subsequently with an energy-consuming and expensive drying step one actually preferably works, however, with the smallest possible amount of liquid, i.e. no lubricant is added other than the amount of liquid introduced via the PTFE dispersion (maximum 40% of the dispersion).

After the kneading step the dough- to fleece-like product is rolled out biaxially between heated rollers (temperature 60-150° C.) in a number of steps to form a mat first of all, and then to form a film, the fibrillation being optimised and a homogeneous final layer thickness of 0.3 to 1 mm, preferably 0.4-0.6 mm being set. A heatable calendar or roller system comprising at least 2 rollers, preferably 4 rollers or more, is suitable for this step.

A suitable method for producing a composite material composed of a polymer a) and a zeolite b) is also described in EP 0 773 829 B1 and the documents cited in the latter.

If a metal material is to be introduced in two-dimensional form, the material thus obtained is pressed in one or more steps between pressure-loaded rollers within a laminator or calendar with the metal material in two-dimensional form, e.g. stainless steel mesh, such that a composite composed of at least one layer of the material and the metal material is formed. Preferably, a layer of the metal material is enclosed between two layers of the material [FIG. 2]. Preferably both layers of the material penetrate through the openings in the two-dimensional metal material, by means of which the stability of the composite is optimised. The step of connecting the metal material and the material can take place at room temperature, advantageously however at 70-250° C., in order to eliminate any residual moisture which may be present in the material, for example, due to the use of water as a lubricant when mixing and/or kneading polymer containing fluorine a) and zeolite particles b) as described above. A drying step optionally follows.

Optionally, one or more heating element(s) is/are introduced into the material such that the heat energy can be easily transferred from the heating element to the metal material.

The metal material can optionally itself perform the function of the heating element e.g. by heating by means of magnetic induction, electric resistance heating or heat exchange. By means of the heating element the adsorption and desorption temperature can be optimised within the framework of the process yield. It serves, moreover, to facilitate the optionally necessary regeneration of the material [FIG. 3].

The composite material according to the first aspect of the invention can be used for the adsorption of organic molecules which are contained in a gaseous or liquid mixture of substances.

### Method for the Adsorption of Organic Molecules

In the following the method will be described according to the second aspect of the invention.
a) Bringing a mixture of substances, that contains at least one type of organic molecules, into contact with the composite material according to the first aspect of the present invention.

The adsorption of at least one organic component, i.e. of at least one type of organic molecules, takes place from a fluid, i.e. from a liquid or gaseous mixture of substances, that contains at least one organic component and preferably water. Hydrogen sulphide, ammonia, hydrogen, carbon dioxide or nitrogen can be present in the fluid as non-organic components.

The at least one organic component is for example a substance composed of one of the substance-class alcohols (e.g. ethanol, butanol), ether (e.g. methyl tert butyl ether or tetrahydrofuran), ketones (e.g. acetone), aldehydes (e.g. acetaldehyde), carboxylic acids (in particular C₁₋₄ carboxylic acids such as e.g. acetic acid or propionic acid) and carboxylic acid ester (e.g. ethyl acetate). These substances are preferably produced fermentatively or enzymatically. This production is particularly preferably an ethanolic fermentation by means of yeasts or bacteria or so-called ABE fermentation by means of bacteria, the latter producing acetone, butanol and ethanol (ABE).

Preferably the fluid that is used for the adsorption is a gaseous mixture of substances that is obtained by gas stripping an aqueous solution with volatile organic compounds of the substance classes specified above. The aqueous solution is particularly preferably a fermentation solution, very particularly preferably a fermentation solution of the fermentation processes specified above. This gas stripping is particularly preferably implemented in situ, in situ meaning that the gas stripping takes place during fermentation. However, the gas stripping can also take place after the fermentation is completed. The gas stripping can take place in an external gas stripping apparatus connected to the fermenter.

The composite material according to the first aspect of the present invention is brought into contact with the mixture of substances so that adsorption of the at least one organic component can take place. For example, the composite material can be introduced into a flow of the gaseous or liquid mixture of substances such that the mixture of substances can flow over the surface of the composite material, for example by arranging the composite material in a suitable geometric form in a column through which a flow of fluid is guided.

By adsorption of the at least one type of organic molecules on the composite material the composite material is charged with the at least one type of organic molecules so that a charged composite material is obtained.
b) The charged composite material is separated from the mixture of substances after the composite material has been in contact with the mixture of substances over a period that is sufficiently long for the adsorption of the at least one organic component in order to achieve sufficient charging of the composite material. If a flow of fluid is guided over the packing of the composite material so that adsorption of at least one organic component contained in the latter occurs, a gradient generally forms for the charge, i.e. the concentration of organic component adsorbed on the composite material in the flow direction. This means that the concentration of organic component adsorbed on the composite material is generally higher in the regions of the composite material lying upstream than in the regions of the composite material lying further downstream.
c) The at least one organic component is separated from the composite material by desorption.

The desorption can take place
(a) by expulsion by means of other components;
(b) thermally, i.e. by increasing the temperature of the adsorption means (temperature swing adsorption method (TSA));
(c) by the so-called pressure swing adsorption method (PSA), i.e. by reducing the pressure;
(d) by a combination of the methods specified in (a) to (c).

In a preferred embodiment of the method according to the invention a flushing gas is used for the desorption. Preferred flushing gases are inert gases, and particularly preferably the flushing gases are air, carbon dioxide, nitrogen, noble gases or mixtures of the latter. In a further embodiment of the method according to the invention the flushing gas contains water. Particularly preferably the temperature of the flushing gas is above the temperature of the composite material.

Preferably, the flow direction during desorption is opposite to the flow direction of the fluid during adsorption, i.e. so that desorption takes place contrary to the gradient of the concentration of the organic component adsorbed on the composite material produced during adsorption.

### BRIEF DESCRIPTION OF THE FIGURES

FIG. 1 is a diagrammatic representation of a composite material composed of PTFE and zeolite (101=zeolite particles, 102=net-like PTFE fibrillae).

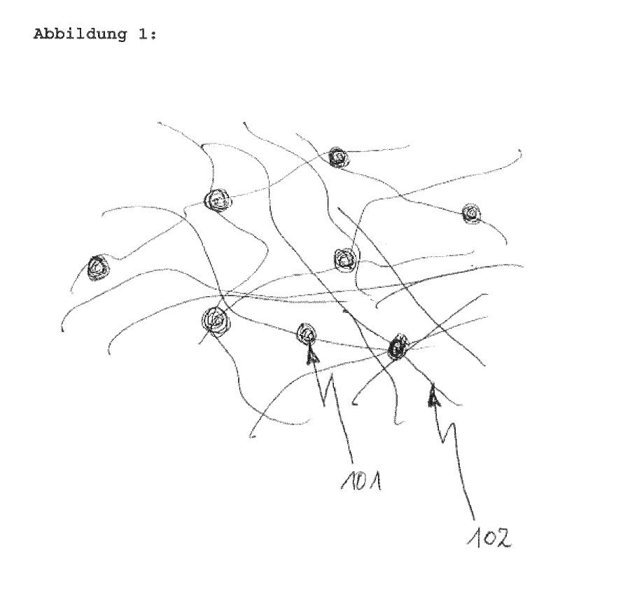

FIG. 2 is a diagrammatic representation of an embodiment of the composite material according to the invention composed of 103=metal material, 110=PTFE zeolite composite according to FIG. 1.

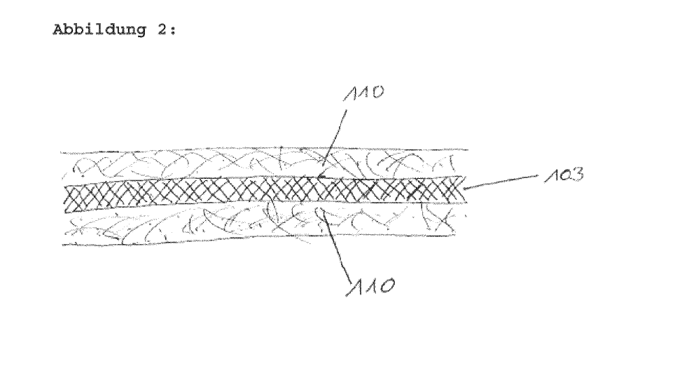

FIG. 3 is a diagrammatic representation of an embodiment of the composite material according to the invention composed of 103=metal material, 110=PTFE zeolite composite according to FIG. 1, 104=heating element.

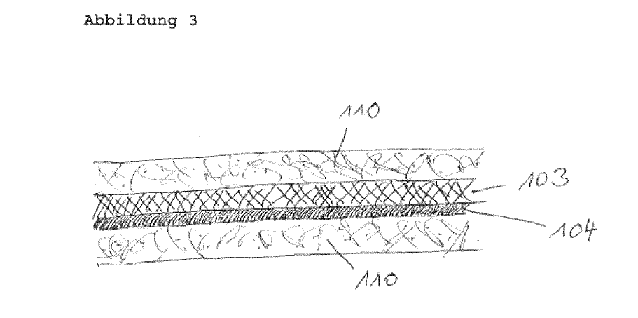

FIG. 4 two shows, as examples, two embodiments of the layer mould bodies of the invention according to FIG. 2.

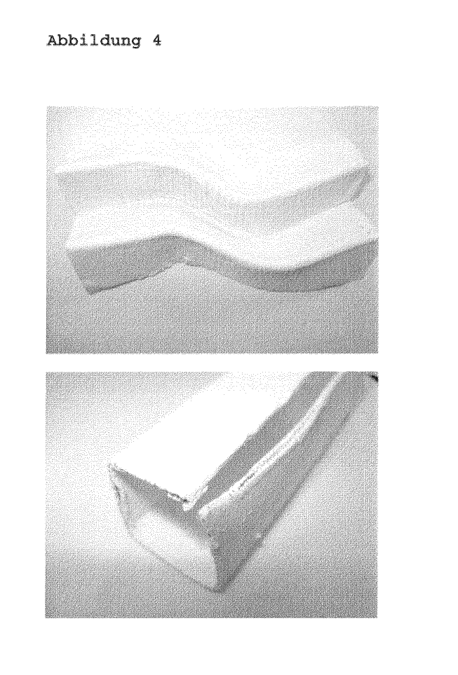

### EXAMPLES

The material according to the invention is described by means of the following non-restrictive examples:

### Example 1

### Production of a PTFE Zeolite Composite Material (Not According to the Invention)

25.1 g PTFE dispersion TE3893-N (approx. 60% PTFE content, DuPont) are mixed with 150 g dried zeolite (ZSM-5, H form; SiO₂/Al₂O₃>800; manufacturer: Süd-Chemie AG, Germany) (corresponding to 9% w/w PTFE) and then kneaded for 45 minutes in a Werner & Pfleiderer LUK 075 laboratory kneader at 90° C., fibrillation of the PTFE and addition of the zeolite ensuing upon shearing.

After the kneading step the fleece-type product is rolled out in a Fetzel calender system between heated rollers (temperature 70°) in a number of steps biaxially to a film with a layer thickness of 0.5 mm, the fibrillation being optimised.

### Example 2

### Production of a Reinforced PTFE Zeolite Composite Material

A layer of stainless steel fabric 20×30 cm (material 1,431, wire thickness 0.25 mm, mesh width 1.00 mm; made by the company Drahtweberei Grafenthal GmbH, Grafenthal) is brought between two layers of film-like material from Example 1 (thickness 0.5 mm, dimensions 20×30 cm), and the three layers are pressed in a laminator made by the company Fetzel (roller gap 0.5 mm; feed rate 1-1.4 m/min) at 60-70° C. in two passages.

### Example 3

Adsorption of Ethanol from a Gas Flow

500 ml of a 5% (w/v) ethanol water solution were stripped for 24 hours with a volumetric flow of 5.5 l/min. With the aid of a water bath (heating plate made by the company IKA; RCT basic, temperature sensor made by the company VWR; type VT5) the solution was tempered to 30° C. A membrane pump (KNF Neuberger, Germany; type: N86KT.18) and a gas washing bottle (VWR, Germany) were used. The gas flow was conveyed through a glass column (made by the company Gassner-Glastechnik, Germany; internal diameter: 6.3 cm; total length 36 cm) which was packed with 155.93 g of the composite material from Example 2 in rolled form. The gas flow was conveyed back into the gas washing bottle within the framework of a circulation process so that the system was closed. The glass column was kept in a climate cabinet (made by the company: Votsch Industrietechnik, Germany; VC 4043) at a temperature of 40° C.

After 24 hours one end of the glass column was closed and the other end was connected by Teflon tubes to two cooling traps in Dewar flasks (made by the company: KGW Isotherm, Germany) which were cooled by liquid nitrogen and finally to a vacuum pump (made by the company: VacuuBrand, Germany; type: CVC3000). The glass column was taken from the climate cabinet and covered with insulating material. The desorption started as soon as the column was connected airtight to the desorption structure. The desorption time lasted 20 minutes at 50 mbar absolute pressure. After the desorption time the experiment was stopped. The volume of the condensed desorbate was determined and the ethanol concentration was measured by gas chromatography.

1. A composite material comprising
a) a porous matrix composed of a polymer containing fluorine having a percentage of tetrafluoroethylene monomer units of at least 95 mol % based on the total of monomer units;
b) hydrophobic zeolite particles which are embedded in the matrix and around which the latter extends, and
c) at least one metal material;
d) optionally at least one further component, wherein:
the amount of metal material c) is 1 to 90% by weight based on the total weight of all of the components,
the ratio of the weight of component a) to the total weight of components b) and d) is 2:98 to 30:70, and
the ratio of the weight of component b) to the weight of component d) is 80:20 to 100:0.

2. The composite material according to claim 1, wherein the polymer containing fluorine is polytetrafluoroethylene.

3. The composite material according to claim 1, wherein the ratio of the weight of component a) to the total weight of components b) and d) is 4:96 to 20:80.

4. The composite material according to claim 1, wherein the ratio of component a) to the total weight of components b) and d) is 5:95 to 15:85.

5. The composite material according to claim 1, wherein the amount of metal material c) is 5 to 80% by weight based on the total weight of all of the components.

6. The composite material according to claim 1, wherein the amount of metal material c) is 10 to 70% by weight based on the total weight of all of the components.

7. The composite material according to claim 1, wherein the metal material is a steel with material number 1.4301.

8. The composite material according to claim 1, wherein the metal material is in the form of a wire fabric, a wire mesh, a plate provided with holes, in the form of shavings or in powder form.

9. The composite material according to claim 1, wherein the metal material is able to be heated electrically, by magnetic induction or by a heat exchange process.

10. The composite material according to claim 1, wherein the zeolite has a particle size of between 0.5 and 100 μm.

11. The composite material according to claim 1, wherein the zeolite is chosen from the group consisting of silicalite, B zeolite, mordenite, Y zeolite, MFI zeolite, ferrierite (FER zeolite), dealuminated, ultra-stable zeolite Y (USY zeolite) and erionite (ERI zeolite) and mixtures of the latter.

12. The composite material according to claim 1, wherein the zeolite has a SiO2/Al2O3 ratio of 100:1 or larger.

13. The composite material according to claim 1, wherein the zeolite has having an SiO2/Al2O3 ratio of 200:1 or larger.

14. A method for adsorption of organic molecules from a gaseous or liquid mixture of substances containing at least one organic component,. the method comprising bringing said mixture of substances into contact with the composite material according to claim 1.

15. A method for removal of organic molecules from a gaseous or liquid mixture of substances containing at least one organic component, the method comprising the following steps:
a) bringing a mixture of substances containing at least one organic component into contact with the composite material according to claim 1 so that the at least one organic component is adsorbed on the composite material and a charged composite material is obtained;
b) separating the mixture of substances and the charged composite material; and
c) desorbing the at least one organic component from the charged composite material.

16. The method according to claim 15, wherein the desorption in step c) is brought about by reducing the pressure of the atmosphere surrounding the composite material and/or increasing the temperature of the composite material.
